(19) United States
(12) Patent Application Publication
(10) Pub. No.: US 2008/0003673 A1
(43) Pub. Date: Jan. 3, 2008

(54) Novel human proteases and polynucleotides encoding the same

(76) Inventors: Alejandro Abuin, The Woodlands, TX (US); Gregory Donoho, Indianapolis, IN (US); Carl Johan Friddle, The Woodlands, TX (US); Glenn Friedrich, Houston, TX (US); Brenda Gerhardt, Spring, TX (US); Erin Hilbun, Denton, TX (US); Yi Hu, Spring, TX (US); James Alvin Kieke, Houston, TX (US); Maricar Miranda, The Woodlands, TX (US); Michael C. Nehls, Stockdorf (DE); Boris Nepomnichy, Dickinson, TX (US); Andrew Olson, San Diego, CA (US); Arthur T. Sands, The Woodlands, TX (US); John Scoville, Pearland, TX (US); C. Alexander Turner JR., The Woodlands, TX (US); D. Wade Walke, Spring, TX (US); Nathaniel L. Wilganowski, Houston, TX (US); Xuanchuan Yu, Conroe, TX (US); Brian Zambrowicz, The Woodlands, TX (US)

Correspondence Address: LEXICON PHARMACEUTICALS, INC., 8800 TECHNOLOGY FOREST PLACE, THE WOODLANDS, TX, 77381-1160, US

(21) Appl. No.: 11/220,398

(22) Filed: Sep. 6, 2005

Related U.S. Application Data
(63) Continuation-in-part of application No. 10/760,709, filed on Jan. 20, 2004, now abandoned.
Application No. 10/760,709 is a division of application No. 10/202,619, filed on Jul. 23, 2002, now Pat. No. 6,716,614.
Application No. 10/202,619 is a continuation-in-part of application No. 09/653,839, filed on Sep. 1, 2000, now Pat. No. 6,433,153.
(63) Continuation-in-part of application No. 10/872,968, filed on Jun. 21, 2004, now abandoned.
Application No. 10/872,968 is a continuation of application No. 10/200,344, filed on Jul. 19, 2002, now Pat. No. 6,780,640.
Application No. 10/200,344 is a continuation of application No. 09/675,305, filed on Sep. 29, 2000, now Pat. No. 6,441,153.
(63) Continuation-in-part of application No. 10/843,130, filed on May 11, 2004, now abandoned.
Application No. 10/843,130 is a division of application No. 10/200,910, filed on Jul. 22, 2002, now Pat. No. 6,777,221.
Application No. 10/200,910 is a continuation of application No. 09/710,099, filed on Nov. 10, 2000, now Pat. No. 6,441,154.
(63) Continuation-in-part of application No. 11/027,743, filed on Dec. 30, 2004, now abandoned.
Application No. 11/027,743 is a continuation of application No. 10/419,276, filed on Apr. 17, 2003, now Pat. No. 6,852,521.
Application No. 10/419,276 is a continuation of application No. 09/963,791, filed on Dec. 8, 2000, now Pat. No. 6,649,399.
(63) Continuation-in-part of application No. 10/889,890, filed on Jul. 12, 2004, now abandoned.
Application No. 10/889,890 is a continuation of application No. 09/735,713, filed on Dec. 12, 2000, now abandoned.
(63) Continuation-in-part of application No. 11/049,613, filed on Feb. 2, 2005, now abandoned.
Application No. 11/049,613 is a continuation of application No. 09/755,016, filed on Jan. 5, 2001, now abandoned.
(63) Continuation-in-part of application No. 11/036,185, filed on Jan. 10, 2005, now abandoned.
Application No. 11/036,185 is a division of application No. 10/766,074, filed on Jan. 28, 2004, now Pat. No. 6,881,563.
Application No. 10/766,074 is a division of application No. 10/214,811, filed on Aug. 7, 2002, now Pat. No. 6,743,621.
Application No. 10/214,811 is a continuation of application No. 09/780,016, filed on Feb. 9, 2001, now Pat. No. 6,509,456.
(63) Continuation-in-part of application No. 10/760,783, filed on Jan. 20, 2004, now abandoned.
Application No. 10/760,783 is a division of application No. 09/784,358, filed on Feb. 15, 2001, now Pat. No. 6,720,412.
(63) Continuation-in-part of application No. 10/984,359, filed on Nov. 8, 2004, now abandoned.
Application No. 10/984,359 is a continuation of application No. 09/833,782, filed on Apr. 12, 2001.
(63) Continuation-in-part of application No. 11/120,146, filed on May 2, 2005, now abandoned.
Application No. 11/120,146 is a continuation of application No. 09/854,844, filed on May 14, 2001, now abandoned.
(63) Continuation-in-part of application No. 10/950,177, filed on Sep. 24, 2004, now abandoned.
Application No. 10/950,177 is a continuation of application No. 09/863,824, filed on May 23, 2001, now abandoned.
(63) Continuation-in-part of application No. 10/804,457, filed on Mar. 19, 2004, now abandoned.
Application No. 10/804,457 is a division of application No. 10/217,774, filed on Aug. 12, 2002, now Pat. No. 6,734,007.
Application No. 10/217,774 is a continuation of application No. 09/930,872, filed on Aug. 15, 2001, now Pat. No. 6,448,388.
(63) Continuation-in-part of application No. 11/039,398, filed on Jan. 20, 2005, now abandoned.
Application No. 11/039,398 is a continuation of application No. 09/938,330, filed on Aug. 22, 2001, now abandoned.
(63) Continuation-in-part of application No. 10/961,020, filed on Oct. 8, 2004, now abandoned.
Application No. 10/961,020 is a continuation of application No. 09/965,631, filed on Sep. 27, 2001, now abandoned.
(63) Continuation-in-part of application No. 09/962,739, filed on Sep. 25, 2001, now abandoned.
(63) Continuation-in-part of application No. 09/969,515, filed on Oct. 2, 2001, now abandoned.
(63) Continuation-in-part of application No. 10/964,106, filed on Oct. 13, 2004, now abandoned.
Application No. 10/964,106 is a continuation of application No. 10/020,733, filed on Oct. 30, 2001, now abandoned.
(63) Continuation-in-part of application No. 10/919,124, filed on Aug. 16, 2004, now abandoned.
Application No. 10/919,124 is a continuation of application No. 10/014,896, filed on Dec. 11, 2001, now abandoned.
(63) Continuation-in-part of application No. 11/049,616, filed on Feb. 2, 2005, now abandoned.
Application No. 11/049,616 is a continuation of application No. 10/022,710, filed on Dec. 13, 2001, now abandoned.
(63) Continuation-in-part of application No. 11/025,651, filed on Dec. 29, 2004, now abandoned.
Application No. 11/025,651 is a continuation of application No. 10/041,770, filed on Jan. 8, 2002, now abandoned.
(63) Continuation-in-part of application No. 10/999,109, filed on Nov. 29, 2004, now abandoned.
Application No. 10/999,109 is a continuation of application No. 10/044,807, filed on Jan. 11, 2002, now abandoned.
(63) Continuation-in-part of application No. 10/078,592, filed on Feb. 19, 2002, now abandoned.
(63) Continuation-in-part of application No. 10/990,935, filed on Nov. 17, 2004, now abandoned.
Application No. 10/990,935 is a continuation of application No. 10/226,560, filed on Aug. 22, 2002, now abandoned.
Application No. 10/226,560 is a continuation-in-part of application No. 09/917,614, filed on Jul. 27, 2001, now abandoned.
(60) Provisional application No. 60/152,057, filed on Sep. 2, 1999.
(60) Provisional application No. 60/156,685, filed on Sep. 29, 1999.
(60) Provisional application No. 60/165,260, filed on Nov. 12, 1999.
(60) Provisional application No. 60/169,769, filed on Dec. 9, 1999.
(60) Provisional application No. 60/171,566, filed on Dec. 22, 1999.
(60) Provisional application No. 60/174,686, filed on Jan. 6, 2000.
(60) Provisional application No. 60/181,924, filed on Feb. 11, 2000.
(60) Provisional application No. 60/183,282, filed on Feb. 17, 2000.
(60) Provisional application No. 60/196,319, filed on Apr. 12, 2000.
(60) Provisional application No. 60/205,275, filed on May 18, 2000.
(60) Provisional application No. 60/206,415, filed on May 23, 2000.
(60) Provisional application No. 60/225,852, filed on Aug. 16, 2000.
(60) Provisional application No. 60/227,104, filed on Aug. 22, 2000.
(60) Provisional application No. 60/233,796, filed on Sep. 19, 2000.
(60) Provisional application No. 60/236,689, filed on Sep. 29, 2000.
(60) Provisional application No. 60/236,690, filed on Sep. 29, 2000.
(60) Provisional application No. 60/237,540, filed on Oct. 4, 2000.
(60) Provisional application No. 60/244,939, filed on Nov. 1, 2000.
(60) Provisional application No. 60/255,567, filed on Dec. 14, 2000.
(60) Provisional application No. 60/259,033, filed on Dec. 28, 2000.
(60) Provisional application No. 60/260,276, filed on Jan. 8, 2001.
(60) Provisional application No. 60/261,684, filed on Jan. 12, 2001.
(60) Provisional application No. 60/270,320, filed on Feb. 20, 2001.
(60) Provisional application No. 60/221,644, filed on Jul. 28, 2000.

(51) Int. Cl. C12N 15/00; C07H 21/04; C07K 1/00

(52) U.S. Cl. 435/320.1; 530/350; 536/23.2

(57) ABSTRACT

Novel human polynucleotide and polypeptide sequences are disclosed that can be used in therapeutic, diagnostic, and pharmacogenomic applications.

### 1.0 CROSS-REFERENCE TO RELATED APPLICATIONS

The present application is a continuation-in-part of: co-pending U.S. application Ser. No. 10/760,709, filed on Jan. 20, 2004, which is a divisional of U.S. application Ser. No. 10/202,619, filed on Jul. 23, 2002, which issued as U.S. Pat. No. 6,716,614 B1 on Apr. 6, 2004, which is a continuation-in-part of U.S. application Ser. No. 09/653,839, filed on Sep. 1, 2000, which issued as U.S. Pat. No. 6,433,153 B1 on Aug. 13, 2002, which claims the benefit of U.S. Provisional Application No. 60/152,057, filed on Sep. 2, 1999; co-pending U.S. application Ser. No. 10/872,968, filed on Jun. 21, 2004, which is a continuation of U.S. application Ser. No. 10/200,344, filed on Jul. 19, 2002, which issued as U.S. Pat. No. 6,780,640 B2 on Aug. 24, 2004, which is a continuation of U.S. application Ser. No. 09/675,305, filed on Sep. 29, 2000, which issued as U.S. Pat. No. 6,441,153 B1 on Aug. 27, 2002, which claims the benefit of U.S. Provisional Application No. 60/156,685, filed on Aug. 29, 1999; co-pending U.S. application Ser. No. 10/843,130, filed on May 11, 2004, which is a divisional of U.S. application Ser. No. 10/200,910, filed on Jul. 22, 2002, which issued as U.S. Pat. No. 6,777,221 B2 on Aug. 17, 2004, which is a continuation of U.S. application Ser. No. 09/710,099, filed on Nov. 10, 2000, which issued as U.S. Pat. No. 6,441,154 B1 on Aug. 27, 2002, which claims the benefit of U.S. Provisional Application No. 60/165,260, filed on Nov. 12, 1999; co-pending U.S. application Ser. No. 11/027,743, filed on Dec. 30, 2004, which is a continuation of U.S. application Ser. No. 10/419,276, filed on Apr. 17, 2003, which issued as U.S. Pat. No. 6,852,521 B2 on Feb. 8, 2005, which is a continuation of U.S. application Ser. No. 09/963,791, filed on Dec. 8, 2000, which issued as U.S. Pat. No. 6,649,399 B2 on Nov. 18, 2003, which claims the benefit of U.S. Provisional Application No. 60/169,769, filed on Dec. 9, 1999; co-pending U.S. application Ser. No. 10/889,890, filed on Jul. 12, 2004, which is a continuation of U.S. application Ser. No. 09/735,713, filed on Dec. 12, 2000, abandoned, which claims the benefit of U.S. Provisional Application No. 60/171,566, filed on Dec. 22, 1999; co-pending U.S. application Ser. No. 11/049,613, filed on Feb. 2, 2005, which is a continuation of U.S. application Ser. No. 09/755,016, filed on Jan. 5, 2001, abandoned, which claims the benefit of U.S. Provisional Application No. 60/174,686, filed on Jan. 6, 2000; co-pending U.S. application Ser. No. 11/036,185, filed on Jan. 10, 2005, which is a divisional of co-pending U.S. application Ser. No. 10/766,074, filed on Jan. 28, 2004, which issued as U.S. Pat. No. 6,881,563 B2 on Apr. 19, 2005, which is a divisional of co-pending U.S. application Ser. No. 10/214,811, filed on Aug. 7, 2002, which issued as U.S. Pat. No. 6,743,621 B2 on Jun. 1, 2004, which is a continuation of U.S. application Ser. No. 09/780,016, filed on Feb. 9, 2001, which issued as U.S. Pat. No. 6,509,456 B2 on Jan. 21, 2003, which claims the benefit of U.S. Provisional Application No. 60/181,924, filed on Feb. 11, 2000; co-pending U.S. application Ser. No. 10/760,783, filed on Jan. 20, 2004, which is a divisional of U.S. application Ser. No. 09/784,358, filed on Feb. 15, 2001, which issued as U.S. Pat. No. 6,720,412 B2 on Apr. 13, 2004, which claims the benefit of U.S. Provisional Application No. 60/183,282, filed on Feb. 17, 2000; co-pending U.S. application Ser. No. 10/984,359, filed on Nov. 8, 2004, which is a continuation of U.S. application Ser. No. 09/833,782, filed on Apr. 12, 2001, which claims the benefit of U.S. Provisional Application No. 60/196,319, filed on Apr. 12, 2000; co-pending U.S. application Ser. No. 11/120,146, filed on May 2, 2005, which is a continuation of U.S. application Ser. No. 09/854,844, filed on May 14, 2001, abandoned, which claims the benefit of U.S. Provisional Application No. 60/205,275, filed on May 18, 2000; co-pending U.S. application Ser. No. 10/950,177, filed on Sep. 24, 2004, which is a continuation of U.S. application Ser. No. 09/863,824, filed on May 23, 2001, abandoned, which claims the benefit of U.S. Provisional Application No. 60/206,415, filed on May 23, 2000; co-pending U.S. application Ser. No. 10/804,457, filed on Mar. 19, 2004, which is a divisional of U.S. application Ser. No. 10/217,774, filed on Aug. 12, 2002, which issued as U.S. Pat. No. 6,734,007 B2 on May 11, 2004, which is a continuation of U.S. application Ser. No. 09/930,872, filed on Aug. 15, 2001, which issued as U.S. Pat. No. 6,448,388 B1 on Sep. 10, 2002, which claims the benefit of U.S. Provisional Application No. 60/225,852, filed on Aug. 16, 2000; co-pending U.S. application Ser. No. 11/039,398, filed on Jan. 20, 2005, which is a continuation of U.S. application Ser. No. 09/938,330, filed on Aug. 22, 2001, abandoned, which claims the benefit of U.S. Provisional Application No. 60/233,796, filed on Sep. 19, 2000, and 60/227,104, filed on Aug. 22, 2000; co-pending U.S. application Ser. No. 10/961,020, filed on Oct. 8, 2004, which is a continuation of U.S. application Ser. No. 09/965,631, filed on Sep. 27, 2001, abandoned, which claims the benefit of U.S. Provisional application No. 60/236,689, filed on Sep. 29, 2000; co-pending U.S. application Ser. No. 09/962,739, filed on Sep. 25, 2001, which claims the benefit of U.S. Provisional Application No. 60/236,690, filed on Sep. 29, 2000; co-pending U.S. application Ser. No. 09/969,515, filed on Oct. 2, 2001, which claims the benefit of U.S. Provisional Application No. 60/237,540, filed on Oct. 4, 2000; co-pending U.S. application Ser. No. 10/964,106, filed on Oct. 13, 2004, which is a continuation of U.S. application Ser. No. 10/020,733, filed on Oct. 30, 2001, abandoned, which claims the benefit of U.S. Provisional Application No. 60/244,939, filed on Nov. 1, 2000; co-pending U.S. application Ser. No. 10/919,124, filed on Aug. 16, 2004, which is a continuation of U.S. application Ser. No. 10/014,896, filed on Dec. 11, 2001, abandoned, which claims the benefit of U.S. Provisional Application No. 60/255,567, filed on Dec. 14, 2000; co-pending U.S. application Ser. No. 11/049,616, filed on Feb. 2, 2005, which is a continuation of U.S. application Ser. No. 10/022,710, filed on Dec. 13, 2001, abandoned, which claims the benefit of U.S. Provisional Application No. 60/259,033, filed on Dec. 28, 2000; co-pending U.S. application Ser. No. 11/025,651, filed on Dec. 29, 2004, which is a continuation of U.S. application Ser. No. 10/041,770, filed on Jan. 8, 2002, abandoned, which claims the benefit of U.S. Provisional Application No. 60/260,276, filed on Jan. 8, 2001; co-pending U.S. application Ser. No. 10/999,109, filed on Nov. 29, 2004, which is a continuation of U.S. application Ser. No. 10/044,807, filed on Jan. 11, 2002, abandoned, which claims the benefit of U.S. Provisional Application No. 60/261,684, filed on Jan. 12, 2001; co-pending U.S. application Ser. No. 10/078,592, filed on Feb. 19, 2002, which claims the benefit of U.S. Provisional Application No. 60/270,320, filed on Feb. 20, 2001; and co-pending U.S. application Ser. No. 10/990,935, filed on Nov. 17, 2004, which is a continuation of U.S. application Ser. No. 10/226,560, filed on Aug. 22, 2002, abandoned, which both claims the benefit of U.S. Provisional Application No. 60/314,049, filed on Aug. 22, 2001, and is a continuation-in-part of U.S. application Ser. No. 09/917,614, filed on Jul. 27, 2001, abandoned, which claims the benefit of U.S. Provisional Application No. 60/221,644, filed on Jul. 28, 2000; each of which is herein incorporated by reference in its entirety.

### 2.0 CROSS-REFERENCE TO SEQUENCE LISTING SUBMITTED ON COMPACT DISC

The present application contains a Sequence Listing of SEQ ID NOS:1-222, in file “SeqList.txt” (1,415,168 bytes), created on Aug. 30, 2005, submitted herewith on duplicate compact disc (Copy 1 and Copy 2), which is herein incorporated by reference in its entirety.

### 3.0 INTRODUCTION

The present invention relates to the discovery, identification, and characterization of novel human polynucleotides encoding proteins sharing sequence similarity with mammalian proteases, neurolysin proteins, and animal proteins having thrombospondin repeats. The invention encompasses the described polynucleotides, host cell expression systems, the encoded proteins, fusion proteins, polypeptides and peptides, antibodies to the encoded proteins and peptides, and genetically engineered animals that either lack or overexpress the disclosed polynucleotides, antagonists and agonists of the proteins, and other compounds that modulate the expression or activity of the proteins encoded by the disclosed polynucleotides, which can be used for diagnosis, drug screening, clinical trial monitoring, the treatment of diseases and disorders, and cosmetic or nutriceutical applications.

### 4.0 BACKGROUND OF THE INVENTION

Proteases are enzymes that mediate the proteolytic cleavage of protein substrates as part of degradation, maturation, and secretory pathways within the body. Proteases have been associated with, inter alia, regulating development, diabetes, obesity, modulating cellular processes, fertility, and infectious disease.

Calcium-dependent proteases, such as calpains, have been found in virtually every vertebrate cell that has been examined for their presence. The calpain system has at least three well-characterized protein members that are activated in response to changes in calcium concentration. These proteins include at least two calpains that are activated at different concentrations of calcium, and a calpastatin that specifically inhibits the two calpains. Various tissue/species specific cDNAs have been described that are homologous to the calpains. Given the near ubiquitous expression of calpains, they have been implicated in a wide variety of cellular functions including, but not limited to, cell proliferation and differentiation, signal transduction, processes involving interactions between the cell membrane and cytoskeleton, secretion, platelet aggregation, cytokinesis, and disease. Accordingly, calpains represent a key target for the regulation of a variety of biological pathways.

Carboxypeptidases are proteases that hydrolyze the peptide bonds at the carboxy-terminal end of a chain of amino acids, and have been identified in a wide variety of cell types and animals. Peptidases have been implicated in a wide variety of biological processes including, but not limited to, digestion, coagulation, diabetes, prostate cancer, gynecological disorders, neurological disorders, and obesity. Peptidases thus represent significant targets for regulatory control of a variety of physiological processes and pathways.

Thrombospondins are membrane bound or extracellular proteins that have been implicated in, inter alia, blood clotting, angiogenesis, diabetes, cell adhesion, inflammation, wound healing, cancer, and development. Proteins having thrombospondin repeats can act as receptors, secreted extracellular matrix proteins, and proteases.

Neurolysins are soluble proteins of the zinc metalloprotease family that bind and cleave protein substrates such as angiotensin or neurotensin (typically between proline and tyrosine residues). As such, neurolysins have been implicated in a number of biological processes and anomalies such as blood pressure regulation, kidney function, pain management, cardiac disease, natriuresis and diabetes.

Thus, proteases are proven drug targets.

### 5.0 SUMMARY OF THE INVENTION

The present invention relates to the discovery, identification, and characterization of nucleotides that encode novel human proteins, and the corresponding amino acid sequences of these proteins. The novel human proteins (NHPs), described for the first time herein, share structural similarity with animal proteases, and particularly: calcium dependent or calcium activated proteases, or calpains (SEQ ID NO:1-9); carboxypeptidases, especially carboxypeptidase B and carboxypeptidase A (SEQ ID NOS:10-22); carboxypeptidases, especially carboxypeptidase A, and particularly A1 or A2 (SEQ ID NOS:23-37); metalloproteinases such as ADAM-TS6 (see, e.g., Hurskainen et al., J. Biol Chem. 274:25555-25563, 1999), a zinc metalloproteinase (however these NHPs contain additional regions (exons) that make them unique) (SEQ ID NOS:38-62); trypsin-like proteases such as oviductin, plasminogen activators, and human plasma kallikrein precursor (SEQ ID NOS:63-69); trypsin-like serine proteases such as enteropeptidase (enterokinase), plasminogen, and acrosin (SEQ ID NOS:70-76); aminopeptidases, particularly aminopeptidase P (SEQ ID NOS:77-103); proteins having thrombospondin repeats, such as proteinases, thrombospondin-1, F-spondin, ADAMTS metalloproteases, Tango-71, and distintegrins (SEQ ID NOS:104-120); neurolysins and angiotensin-binding proteins (SEQ ID NOS:121-123); serine proteases (SEQ ID NOS:124-126); thrombospondins (via tsp1 repeats), semaphorins, metalloproteinases, and a serine palmitoyltransferase (SEQ ID NOS:127-132); metalloproteinases (especially zinc metalloproteases of the ADAMTS family), thrombospondin repeat proteins, disintegrins, and aggrecanases (SEQ ID NOS:133-137); metalloproteinases (especially zinc metalloproteases of the ADAMTS family), proteases having thrombospondin repeats, disintegrins, aggrecanases, and procollagen I N-proteinase (SEQ ID NOS:138-170 and 176-188); calpains and calcium-dependant neutral proteases (SEQ ID NOS:171-175); meltrin-beta and ADAM 19 (SEQ ID NOS:189-197); carboxypeptidases, as well as aminoacylases, desuccinylases, deacetylases, and amidohydrolases (SEQ ID NOS:198-201); membrane proteins containing multiple thrombospondin repeats, such as cell adhesion proteins, as well as semaphorins, and a variety of cell surface markers and receptors (SEQ ID NOS:202-208); matrix metalloproteases, zinc dependent metalloproteases, and the ADAMTS family of secreted proteases (SEQ ID NOS:209-211); matrix metalloproteases, zinc dependent metalloproteases, ADAMTS family metalloproteases, collagenases, as well as receptor-linked phosphatases and membrane associated cell adhesion proteins (SEQ ID NOS:212 and 213); matrix metalloproteases, zinc dependent metalloproteases, and bone morphogenetic protein (SEQ ID NOS:214-216); and disintegrins and zinc metalloproteases of the ADAM family, more particularly those of the ADAM-TS family (SEQ ID NO:217-222). Accordingly, the described NHPs encode novel protease proteins with a range of homologues and orthologs that transcend phyla and a broad range of species.

The novel human nucleic acid (cDNA) sequences described herein encode proteins/open reading frames (ORFs) of 739, 723, 702, 686, 47, 88, 247, 92, 437, 350, 351, 314, 436, 399, 351, 314, 69, 908, 292, 468, 310, 507, 589, 141, 317, 159, 356, 438, 757, 306, 302, 164, 217, 348, 288, 507, 69, 290, 265, 211, 267, 186, 242, 453, 532, 428, 509, 484, 1691, 446, 372, 724, 650, 845, 771, 1617, 704, 346, 464, 164, 311, 491, 1224, 451, 297, 486, 1222, 1219, 1216, 1213, 1235, 1232, 1252, 1249, 1907, 321, 950, 367, 669, 353, 1224, 980, 476, 1213, 969, 465, 926, 918, 963, 955, 502, 361, 1465, 1590, 1606, 877, 1762, 436, 862, and 509 amino acids in length (SEQ ID NOS:2, 4, 6, 8, 11, 13, 15, 17, 19, 21, 24, 26, 28, 30, 32, 34, 36, 39, 41, 43, 45, 47, 49, 51, 53, 55, 57, 59, 61, 64, 66, 68, 71, 73, 75, 78, 80, 82, 84, 86, 88, 90, 92, 94, 96, 98, 100, 102, 105, 107, 109, 111, 113, 115, 117, 119, 122, 125, 128, 130, 132, 134, 136, 139, 141, 143, 145, 147, 149, 151, 153, 155, 157, 159, 162, 165, 167, 169, 172, 174, 177, 179, 181, 183, 185, 187, 190, 192, 194, 196, 199, 201, 203, 205, 207, 210, 213, 215, 218, and 221, respectively). SEQ ID NOS:9, 22, 37, 62, 69, 76, 103, 120, 123, 126, 137, 160, 163, 170, 175, 188, 197, 208, 211, 216, 219, and 222 describe NHP ORFs and flanking regions.

The invention also encompasses agonists and antagonists of the described NHPs, including small molecules, large molecules, mutant NHPs (or portions thereof) that compete with native NHPs, peptides, and antibodies, as well as nucleotide sequences that can be used to inhibit the expression of the described NHPs (e.g., antisense and ribozyme molecules, and open reading frame or regulatory sequence replacement constructs) or to enhance the expression of the described NHPs (e.g., expression constructs that place the described polynucleotide under the control of a strong promoter system), and transgenic animals that express an NHP sequence, or “knock-outs” (which can be conditional) that do not express a functional NHP. Knock-out mice can be produced in several ways, one of which involves the use of mouse embryonic stem cell (“ES cell”) lines that contain gene trap mutations in a murine homolog of at least one of the described NHPs. When the unique NHP sequences described in SEQ ID NOS:1-222 are “knocked-out” they provide a method of identifying phenotypic expression of the particular gene, as well as a method of assigning function to previously unknown genes. In addition, animals in which the unique NHP sequences described in SEQ ID NOS:1-222 are “knocked-out” provide an unique source in which to elicit antibodies to homologous and orthologous proteins, which would have been previously viewed by the immune system as “self” and therefore would have failed to elicit significant antibody responses.

Additionally, the unique NHP sequences described in SEQ ID NOS:1-222 are useful for the identification of protein coding sequences, and mapping an unique gene to a particular chromosome. These sequences identify biologically verified exon splice junctions, as opposed to splice junctions that may have been bioinformatically predicted from genomic sequence alone. The sequences of the present invention are also useful as additional DNA markers for restriction fragment length polymorphism (RFLP) analysis, and in forensic biology, particularly given the presence of nucleotide polymorphisms within the described sequences.

Further, the present invention also relates to processes for identifying compounds that modulate, i.e., act as agonists or antagonists of, NHP expression and/or NHP activity that utilize purified preparations of the described NHPs and/or NHP products, or cells expressing the same. Such compounds can be used as therapeutic agents for the treatment of any of a wide variety of symptoms associated with biological disorders or imbalances.

### 6.0 BRIEF DESCRIPTION OF THE FIGURES

No Figures are required in the present invention.

### 7.0 DETAILED DESCRIPTION OF THE INVENTION

The NHPs described for the first time herein are novel proteins that are expressed in, inter alia, human cell lines, and: human prostate and testis cells (SEQ ID NOS:1-9); human brain, pituitary, spinal cord, thymus, spleen, lymph node, bone marrow, trachea, lung, kidney, prostate, testis, thyroid, adrenal gland, stomach, small intestine colon, skeletal muscle, uterus, mammary gland, bladder, and cervix cells (SEQ ID NOS:10-22); human prostate, testis, and placenta cells (SEQ ID NOS:23-37); human fetal brain, brain, thymus, spleen, lymph node, trachea, kidney, fetal liver, testis, thyroid, adrenal gland, stomach, small intestine, uterus, placenta, adipose, esophagus, bladder, cervix, rectum, pericardium, ovary, and fetal lung cells (SEQ ID NOS:38-62); human thymus, trachea, kidney, prostate, testis, thyroid, salivary gland, stomach, placenta, mammary gland, adipose, skin, esophagus, bladder, pericardium, and fetal kidney cells (SEQ ID NOS:63-69); human testis cells (SEQ ID NOS:70-76); human fetal brain, brain, pituitary, cerebellum, spinal cord, thymus, spleen, lymph node, bone marrow, trachea, kidney, fetal liver, liver, prostate, testis, thyroid, adrenal gland, pancreas, salivary gland, stomach, small intestine, colon, uterus, placenta, mammary gland, adipose, skin, esophagus, bladder, cervix, rectum, pericardium, hypothalamus, ovary, fetal kidney, and fetal lung cells (SEQ ID NOS:77-103); human pituitary, lymph node, prostate, testis, adrenal gland, uterus, fetal kidney, fetal lung, and gene trapped human cells (SEQ ID NOS:104-120); human fetal brain, brain, cerebellum, spinal cord, thymus, trachea, kidney, fetal liver, liver, prostate, testis, adrenal gland, pancreas, salivary gland, stomach, small intestine, colon, skeletal muscle, uterus, mammary gland, esophagus, bladder, cervix, rectum, pericardium, hypothalamus, and gene trapped human cell lines (SEQ ID NOS:121-123); human brain, cerebellum, spinal cord, thymus, kidney, testis, adrenal gland, salivary gland, skeletal muscle, and gene trapped human cells (SEQ ID NOS:124-126); human brain, fetal brain, pituitary, cerebellum, spinal cord, thymus, spleen, trachea, kidney, liver, thyroid, adrenal gland, salivary gland, heart, uterus, stomach, small intestine, placenta, mammary gland, adipose, skin, esophagus, cervix, pericardium, fetal lung, and gene trapped human cells (SEQ ID NOS:127-132); human fetal brain, brain, pituitary, cerebellum, spinal cord, thymus, lymph node, trachea, kidney, fetal liver, prostate, testis, thyroid, adrenal gland, pancreas, small intestine, colon, skeletal muscle, heart, uterus, mammary gland, adipose, esophagus, bladder, cervix, pericardium, ovary, fetal kidney, and fetal lung cells (SEQ ID NOS:133-137); human spinal cord, lymph node, bone marrow, trachea, mammary gland, skeletal muscle, pericardium, adipose, esophagus, bladder, fetal kidney, and fetal lung cells (SEQ ID NOS:138-160); human heart, fetal kidney and fetal lung cells (SEQ ID NOS:161-163); human fetal brain, pituitary, cerebellum, spinal cord, lymph node, kidney, fetal liver, liver, prostate, testis, thyroid, adrenal gland, stomach, small intestine, colon, mammary gland, skeletal muscle, heart, uterus, placenta, pericardium, adipose, esophagus, cervix, rectum, ovary, fetal kidney, and fetal lung cells (SEQ ID NOS:164-170); human brain, trachea, kidney, prostate, testis, pancreas, stomach, small intestine, colon, skeletal muscle, mammary gland, adipose, esophagus, cervix, rectum, pericardium, hypothalamus, lymph node, fetal kidney, and fetal lung cells (SEQ ID NOS:171-175); human fetal brain, brain, pituitary, kidney, fetal liver, liver, prostate, testis, thyroid, adrenal gland, salivary gland, stomach, small intestine, colon, skeletal muscle, heart, placenta, mammary gland, adipose, esophagus, trachea, cervix, rectum, pericardium, hypothalamus, ovary, fetal kidney, and fetal lung cells (SEQ ID NOS:176-188); human fetal brain, brain, pituitary, cerebellum, spinal cord, thymus, spleen, lymph node, bone marrow, trachea, lung, kidney, fetal liver, liver, prostate, testis, adrenal gland, pancreas, salivary gland, stomach, small intestine, colon, skeletal muscle, heart, uterus, placenta, mammary gland, skin, adipose, esophagus, bladder, cervix, rectum, hypothalamus, ovary, fetal kidney, gall bladder, tongue, carcinoma cells, umbilical vein, endothelium, and fetal lung cells (SEQ ID NOS:189-197); human fetal brain, pituitary, thymus, lymph node, kidney, testis, adrenal gland, pancreas, placenta, skin, fetal kidney, fetal lung, and 9 week old embryo cells (SEQ ID NOS:198-201); human fetal brain, pituitary gland, spinal cord, thymus, lymph node, bone marrow, trachea, lung, kidney, fetal liver, liver, prostate, testis, thyroid, adrenal gland, stomach, small intestine, colon, skeletal muscle, uterus, placenta, mammary gland, adipose, skin, bladder, pericardium, ovary, fetal kidney, fetal lung, gall bladder, tongue, 6- and 9-week old embryos, and embryonic carcinoma cells (SEQ ID NOS:202-208); human pituitary, lymph node, bone marrow, small intestine, colon, skeletal muscle, uterus, placenta, mammary gland, bladder, cervix, fetal kidney, fetal lung, 12-week old embryos, adenocarcinoma, and osteosarcoma cells (SEQ ID NOS:209-211); human fetal brain, brain, pituitary, cerebellum, spinal cord, thymus, lymph node, trachea, lung, kidney, fetal liver, prostate, testis, thyroid, adrenal gland, stomach, small intestine, colon, skeletal muscle, heart, uterus, placenta, mammary gland, adipose, skin, esophagus, bladder, cervix, rectum, pericardium, ovary, fetal kidney, fetal lung, gall bladder, aorta, 6-, 9-, and 12-week old embryos, osteosarcoma, umbilical vein, and microvascular endothelial cells (SEQ ID NOS:212 and 213); human lymph node, mammary gland, and fetal kidney cells (SEQ ID NOS:214-216); and human pituitary and cerebellum (SEQ ID NOS:217-222).

The described sequences were compiled from: gene trapped cDNAs and clones isolated from a human testis cDNA library (SEQ ID NOS:1-9); gene trapped cDNAs and a clone isolated from a human prostate cDNA library (SEQ ID NOS:10-22); gene trapped cDNAs and clones isolated from a human testis cDNA library (SEQ ID NOS:23-37); gene trapped cDNAs and clones isolated from human testis and placenta cDNA libraries (SEQ ID NOS:38-62); gene trapped cDNAs and clones isolated from a human kidney cDNA library (SEQ ID NOS:63-69); gene trapped cDNAs and clones isolated from a human testis cDNA library (SEQ ID NOS:70-76); gene trapped cDNAs and clones isolated from a human testis cDNA library (SEQ ID NOS:77-103); clustered human gene trapped sequences, ESTs, and cDNA isolated from human lymph node, pituitary, placenta, trachea and mammary gland cDNA cell libraries (SEQ ID NOS:104-120); by aligning human EST sequences and cDNA clones from a HUVEC cDNA library (SEQ ID NOS:121-123); gene trapped cDNAs and cDNAs prepared and isolated from human brain, cerebellum, testis, kidney, skeletal muscle, thymus, and salivary gland mRNA (SEQ ID NOS:124-126); clustered human gene trapped sequences, and cDNA products isolated from human skeletal muscle, mammary gland, uterus, and kidney mRNAs (SEQ ID NOS:127-132); cDNA clones, genomic sequence, and cDNAs derived from human kidney, mammary gland, and cerebellum mRNAs (SEQ ID NOS:133-137); cDNA clones, genomic sequence, and cDNAs derived from human lymph node, thyroid, fetal brain, bone marrow, trachea, kidney, and mammary gland mRNAs (SEQ ID NOS:138-163); cDNA clones, genomic sequence, and cDNAs derived from human kidney, lymph node, pituitary, and thymus mRNAs (SEQ ID NOS:164-170); cDNA clones, genomic sequence, and cDNAs derived from human kidney and testis mRNAs (SEQ ID NOS:171-175); sequence tags, genomic sequence, and cDNAs derived from human placenta, fetal tissue, prostate, thymus, and uterus mRNAs (SEQ ID NOS:176-188); cDNA clones, genomic sequence, and cDNAs derived from human fetal brain, testis, mammary gland, placenta, adipose, uterus, skeletal muscle, fetus, kidney, brain, thymus, and adrenal gland mRNAs (SEQ ID NOS:189-197); cDNA clones, genomic sequence, and cDNAs derived from human thymus, kidney, and lymph node mRNAs (SEQ ID NOS:198-201); genomic sequence and cDNAs from human testis, pituitary, mammary gland, placenta, thymus, fetus, and skeletal muscle mRNAs (SEQ ID NOS:202-208); cDNAs prepared and isolated from human adrenal gland and placenta mRNAs (SEQ ID NOS:209-211); cDNAs prepared and isolated from human lymph node, kidney, and prostate mRNAs (SEQ ID NOS:212 and 213); cDNAs prepared and isolated from human lymph node, mammary gland, and brain mRNAs (SEQ ID NOS:214-216); and cDNAs prepared and isolated from brain mRNA (SEQ ID NOS:217-222). The cDNA libraries were purchased from Clontech (Palo Alto, Calif.) and/or Edge Biosystems (Gaithersburg, Md.).

Because of their medical importance, aminoopeptidases, carboxypeptidases, disintegrins, thrombospondins, proteases and metalloproteases similar to the described NHPs have been studied by others, as exemplified in U.S. Pat. Nos. 5,922,546, 5,593,674, 5,155,038, 5,981,222, 6,013,781, 5,972,680, and 5,656,603 (chemical antagonists of aminopeptidase P), which further describe a variety of uses that are also applicable to the described NHPs.

The present invention encompasses the nucleotides presented in the Sequence Listing, host cells expressing such nucleotides, the expression products of such nucleotides, and: (a) nucleotides that encode mammalian homologs of the described nucleotides, including the specifically described NHPs, and the NHP products; (b) nucleotides that encode one or more portions of the NHPs that correspond to functional domains, and the polypeptide products specified by such nucleotide sequences, including, but not limited to, the novel regions of any active domain(s); (c) isolated nucleotides that encode mutant versions, engineered or naturally occurring, of the described NHPs, in which all or a part of at least one domain is deleted or altered, and the polypeptide products specified by such nucleotide sequences, including, but not limited to, soluble proteins and peptides in which all or a portion of the signal sequence is deleted; (d) nucleotides that encode chimeric fusion proteins containing all or a portion of a coding region of an NHP, or one of its domains (e.g., a receptor or ligand binding domain, accessory protein/self-association domain, etc.) fused to another peptide or polypeptide; or (e) therapeutic or diagnostic derivatives of the described polynucleotides, such as oligonucleotides, antisense polynucleotides, ribozymes, dsRNA, or gene therapy constructs comprising a sequence first disclosed in the Sequence Listing.

As discussed above, the present invention includes the human DNA sequences presented in the Sequence Listing (and vectors comprising the same), and additionally contemplates any nucleotide sequence encoding a contiguous NHP open reading frame (ORF) that hybridizes to a complement of a DNA sequence presented in the Sequence Listing under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (“Current Protocols in Molecular Biology”, Vol. 1, p. 2.10.3 (Ausubel et al., eds., Green Publishing Associates, Inc., and John Wiley & Sons, Inc., New York, 1989)) and encodes a functionally equivalent expression product. Additionally contemplated are any nucleotide sequences that hybridize to the complement of a DNA sequence that encodes and expresses an amino acid sequence presented in the Sequence Listing under moderately stringent conditions, e.g., washing in 0.2×SSC/0.1% SDS at 42° C. (“Current Protocols in Molecular Biology”, supra), yet still encode a functionally equivalent NHP product. Functional equivalents of the NHPs include naturally occurring NHPs present in other species, and mutant NHPs, whether naturally occurring or engineered (by site directed mutagenesis, gene shuffling, directed evolution as described in, for example, U.S. Pat. Nos. 5,837,458 and 5,830,721). The invention also includes degenerate nucleic acid variants of the disclosed NHP polynucleotide sequences.

Additionally contemplated are polynucleotides encoding an NHP ORF, or its functional equivalent, encoded by a polynucleotide sequence that is about 99, 95, 90, or about 85 percent similar or identical to corresponding regions of the nucleotide sequences of the Sequence Listing (as measured by BLAST sequence comparison analysis using, for example, the GCG sequence analysis package (the University of Wisconsin GCG sequence analysis package, SEQUENCHER 3.0, Gene Codes Corp., Ann Arbor, Mich.) using default parameters).

The invention also includes nucleic acid molecules, preferably DNA molecules, that hybridize to, and are therefore the complements of, the described NHP nucleotide sequences. Such hybridization conditions may be highly stringent or less highly stringent, as described herein. In instances where the nucleic acid molecules are deoxyoligonucleotides (“DNA oligos”), such molecules are generally about 16 to about 100 bases long, or about 20 to about 80 bases long, or about 34 to about 45 bases long, or any variation or combination of sizes represented therein that incorporate a contiguous region of sequence first disclosed in the Sequence Listing. Such oligonucleotides can be used in conjunction with the polymerase chain reaction (PCR) to screen libraries, isolate clones, and prepare cloning and sequencing templates, etc.

Alternatively, such NHP oligonucleotides can be used as hybridization probes for screening libraries, and assessing gene expression patterns (particularly using a microarray or high-throughput “chip” format). Additionally, a series of NHP oligonucleotide sequences, or the complements thereof, can be used to represent all or a portion of the described NHP sequences. An oligonucleotide or polynucleotide sequence first disclosed in at least a portion of one or more of the sequences of SEQ ID NOS:1-222 can be used as a hybridization probe in conjunction with a solid support matrix/substrate (resins, beads, membranes, plastics, polymers, metal or metallized substrates, crystalline or polycrystalline substrates, etc.). Of particular note are spatially addressable arrays (i.e., gene chips, microtiter plates, etc.) of oligonucleotides and polynucleotides, or corresponding oligopeptides and polypeptides, wherein at least one of the biopolymers present on the spatially addressable array comprises an oligonucleotide or polynucleotide sequence first disclosed in at least one of the sequences of SEQ ID NOS:1-222, or an amino acid sequence encoded thereby. Methods for attaching biopolymers to, or synthesizing biopolymers on, solid support matrices, and conducting binding studies thereon, are disclosed in, inter alia, U.S. Pat. Nos. 5,700,637, 5,556,752, 5,744,305, 4,631,211, 5,445,934, 5,252,743, 4,713,326, 5,424,186, and 4,689,405.

Addressable arrays comprising sequences first disclosed in SEQ ID NOS:1-222 can be used to identify and characterize the temporal and tissue specific expression of a gene. These addressable arrays incorporate oligonucleotide sequences of sufficient length to confer the required specificity, yet be within the limitations of the production technology. The length of these probes is usually within a range of between about 8 to about 2000 nucleotides. Preferably the probes consist of 60 nucleotides, and more preferably 25 nucleotides, from the sequences first disclosed in SEQ ID NOS:1-222.

For example, a series of NHP oligonucleotide sequences, or the complements thereof, can be used in chip format to represent all or a portion of the described sequences. The oligonucleotides, typically between about 16 to about 40 (or any whole number within the stated range) nucleotides in length, can partially overlap each other, and/or the sequence may be represented using oligonucleotides that do not overlap. Accordingly, the described polynucleotide sequences shall typically comprise at least about two or three distinct oligonucleotide sequences of at least about 8 nucleotides in length that are each first disclosed in the described Sequence Listing. Such oligonucleotide sequences can begin at any nucleotide present within a sequence in the Sequence Listing, and proceed in either a sense (5′-to-3′) orientation vis-a-vis the described sequence or in an antisense orientation.

Microarray-based analysis allows the discovery of broad patterns of genetic activity, providing new understanding of gene functions, and generating novel and unexpected insight into transcriptional processes and biological mechanisms. The use of addressable arrays comprising sequences first disclosed in SEQ ID NOS:1-222 provides detailed information about transcriptional changes involved in a specific pathway, potentially leading to the identification of novel components, or gene functions that manifest themselves as novel phenotypes.

Probes consisting of sequences first disclosed in SEQ ID NOS:1-222 can also be used in the identification, selection, and validation of novel molecular targets for drug discovery. The use of these unique sequences permits the direct confirmation of drug targets, and recognition of drug dependent changes in gene expression that are modulated through pathways distinct from the intended target of the drug. These unique sequences therefore also have utility in defining and monitoring both drug action and toxicity.

As an example of utility, the sequences first disclosed in SEQ ID NOS:1-222 can be utilized in microarrays, or other assay formats, to screen collections of genetic material from patients who have a particular medical condition. These investigations can also be carried out using the sequences first disclosed in SEQ ID NOS:1-222 in silico, and by comparing previously collected genetic databases and the disclosed sequences using computer software known to those in the art.

Thus the sequences first disclosed in SEQ ID NOS:1-222 can be used to identify mutations associated with a particular disease, and also in diagnostic or prognostic assays.

Although the presently described sequences have been specifically described using nucleotide sequence, it should be appreciated that each of the sequences can uniquely be described using any of a wide variety of additional structural attributes, or combinations thereof. For example, a given sequence can be described by the net composition of the nucleotides present within a given region of the sequence, in conjunction with the presence of one or more specific oligonucleotide sequence(s) first disclosed in SEQ ID NOS:1-222. Alternatively, a restriction map specifying the relative positions of restriction endonuclease digestion sites, or various palindromic or other specific oligonucleotide sequences, can be used to structurally describe a given sequence. Such restriction maps, which are typically generated by widely available computer programs (e.g., the University of Wisconsin GCG sequence analysis package, SEQUENCHER 3.0, Gene Codes Corp., etc.), can optionally be used in conjunction with one or more discrete nucleotide sequence(s) present in the sequence that can be described by the relative position of the sequence relative to one or more additional sequence(s) or one or more restriction sites present in the disclosed sequence.

For oligonucleotide probes, highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos). These nucleic acid molecules may encode or act as NHP antisense molecules, useful, for example, in NHP gene regulation and/or as antisense primers in amplification reactions of NHP nucleic acid sequences. With respect to NHP gene regulation, such techniques can be used to regulate biological functions. Further, such sequences may be used as part of ribozyme and/or triple helix sequences that are also useful for NHP gene regulation.

Inhibitory antisense or double stranded oligonucleotides can additionally comprise at least one modified base moiety that is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, (acp3)w, and 2,6-diaminopurine.

The antisense oligonucleotide can also comprise at least one modified sugar moiety selected from the group including, but not limited to, arabinose, 2-fluoroarabinose, xylulose, and hexose.

In yet another embodiment, the antisense oligonucleotide will comprise at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., Nucl. Acids Res. 15:6625-6641, 1987). The oligonucleotide is a 2′-0-methylribonucleotide (Inoue et al., Nucl. Acids Res. 15:6131-6148, 1987), or a chimeric RNA-DNA analogue (Inoue et al., FEBS Lett. 215:327-330, 1987). Alternatively, double stranded RNA can be used to disrupt the expression and function of a targeted NHP.

Oligonucleotides of the invention can be synthesized by standard methods known in the art, e.g., by use of an automated DNA synthesizer (such as are commercially available from Biosearch, Applied Biosystems, etc.). As examples, phosphorothioate oligonucleotides can be synthesized by the method of Stein et al. (Nucl. Acids Res. 16:3209-3221, 1988), and methylphosphonate oligonucleotides can be prepared by use of controlled pore glass polymer supports (Sarin et al., Proc. Natl. Acad. Sci. USA 85:7448-7451, 1988), etc.

Low stringency conditions are well-known to those of skill in the art, and will vary predictably depending on the specific organisms from which the library and the labeled sequences are derived. For guidance regarding such conditions, see, for example, “Molecular Cloning, A Laboratory Manual” (Sambrook et al., eds., Cold Spring Harbor Press, Cold Spring Harbor, N.Y., 1989), “Current Protocols in Molecular Biology”, supra, and periodic updates thereof.

Alternatively, suitably labeled NHP nucleotide probes can be used to screen a human genomic library using appropriately stringent conditions or by PCR. The identification and characterization of human genomic clones is helpful for identifying polymorphisms (including, but not limited to, nucleotide repeats, microsatellite alleles, single nucleotide polymorphisms, or coding single nucleotide polymorphisms), determining the genomic structure of a given locus/allele, and designing diagnostic tests. For example, sequences derived from regions adjacent to the intron/exon boundaries of the human gene can be used to design primers for use in amplification assays to detect mutations within the exons, introns, splice sites (e.g., splice acceptor and/or donor sites), etc., that can be used in diagnostics and pharmacogenomics.

For example, the present sequences can be used in restriction fragment length polymorphism (RFLP) analysis to identify specific individuals. In this technique, an individual's genomic DNA is digested with one or more restriction enzymes, and probed on a Southern blot to yield unique bands for identification (as generally described in U.S. Pat. No. 5,272,057). In addition, the sequences of the present invention can be used to provide polynucleotide reagents, e.g., PCR primers, targeted to specific loci in the human genome, which can enhance the reliability of DNA-based forensic identifications by, for example, providing another “identification marker” (i.e., another DNA sequence that is unique to a particular individual). Actual base sequence information can be used for identification as an accurate alternative to patterns formed by restriction enzyme generated fragments.

Further, an NHP homolog can be isolated from nucleic acid from an organism of interest by performing PCR using two degenerate or “wobble” oligonucleotide primer pools designed on the basis of amino acid sequences within the NHP products disclosed herein. The template for the reaction may be genomic DNA, or total RNA, mRNA, and/or cDNA obtained by reverse transcription of mRNA, prepared from human or non-human cell lines or tissue known to express, or suspected of expressing, an allele of an NHP gene. The PCR product can be subcloned and sequenced to ensure that the amplified sequences represent the sequence of the desired NHP gene. The PCR fragment can then be used to isolate a full length cDNA clone by a variety of methods. For example, the amplified fragment can be labeled and used to screen a cDNA library, such as a bacteriophage cDNA library. Alternatively, the labeled fragment can be used to isolate genomic clones via the screening of a genomic library.

PCR technology can also be used to isolate full length cDNA sequences. For example, RNA can be isolated, following standard procedures, from an appropriate cellular or tissue source (i.e., one known to express, or suspected of expressing, an NHP gene, such as, for example, testis, kidney, lymph node, pituitary or brain tissue). A reverse transcription (RT) reaction can be performed on the RNA using an oligonucleotide primer specific for the most 5′ end of the amplified fragment for the priming of first strand synthesis. The resulting RNA/DNA hybrid may then be “tailed” using a standard terminal transferase reaction, the hybrid may be digested with RNase H, and second strand synthesis may then be primed with a complementary primer. Thus, cDNA sequences upstream of the amplified fragment can be isolated. For a review of cloning strategies that can be used, see, e.g., “Molecular Cloning, A Laboratory Manual”, supra.

A cDNA encoding a mutant NHP sequence can be isolated, for example, by using PCR. In this case, the first cDNA strand may be synthesized by hybridizing an oligo-dT oligonucleotide to mRNA isolated from tissue known to express, or suspected of expressing, an NHP, in an individual putatively carrying a mutant NHP allele, and by extending the new strand with reverse transcriptase. The second strand of the cDNA is then synthesized using an oligonucleotide that hybridizes specifically to the 5′ end of the normal sequence. Using these two primers, the product is then amplified via PCR, optionally cloned into a suitable vector, and subjected to DNA sequence analysis through methods well-known to those of skill in the art. By comparing the DNA sequence of the mutant NHP allele to that of a corresponding normal NHP allele, the mutation(s) responsible for the loss or alteration of function of the mutant NHP gene product can be ascertained.

Alternatively, a genomic library can be constructed using DNA obtained from an individual suspected of carrying, or known to carry, a mutant NHP allele (e.g., a person manifesting an NHP-associated phenotype such as, for example, altered white blood cell levels, obesity, vision disorders, high blood pressure, connective tissue disorders, arthritis, restenosis, behavioral disorders, colitis or spastic colon, asthma, depression, infertility, etc.), or a cDNA library can be constructed using RNA from a tissue known to express, or suspected of expressing, a mutant NHP allele. A normal NHP gene, or any suitable fragment thereof, can then be labeled and used as a probe to identify the corresponding mutant NHP allele in such libraries. Clones containing mutant NHP sequences can then be purified and subjected to sequence analysis according to methods well-known to those skilled in the art.

Additionally, an expression library can be constructed utilizing cDNA synthesized from, for example, RNA isolated from a tissue known to express, or suspected of expressing, a mutant NHP allele in an individual suspected of carrying, or known to carry, such a mutant allele. In this manner, gene products made by the putatively mutant tissue can be expressed and screened using standard antibody screening techniques in conjunction with antibodies raised against a normal NHP product, as described below (for screening techniques, see, for example, “Antibodies: A Laboratory Manual” (Harlow and Lane, eds., Cold Spring Harbor Press, Cold Spring Harbor, N.Y., 1988)).

Additionally, screening can be accomplished by screening with labeled NHP fusion proteins, such as, for example, alkaline phosphatase-NHP or NHP-alkaline phosphatase fusion proteins. In cases where an NHP mutation results in an expression product with altered function (e.g., as a result of a missense or a frameshift mutation), polyclonal antibodies to an NHP are likely to cross-react with a corresponding mutant NHP expression product. Library clones detected via their reaction with such labeled antibodies can be purified and subjected to sequence analysis according to methods well-known in the art.

The invention also encompasses: (a) DNA vectors that contain any of the foregoing NHP coding sequences and/or their complements (i.e., antisense); (b) DNA expression vectors that contain any of the foregoing NHP coding sequences operatively associated with a regulatory element that directs the expression of the coding sequences (for example, baculovirus as described in U.S. Pat. No. 5,869,336); (c) genetically engineered host cells that contain any of the foregoing NHP coding sequences operatively associated with a regulatory element that directs the expression of the coding sequences in the host cell; and (d) genetically engineered host cells that express an endogenous NHP sequence under the control of an exogenously introduced regulatory element (i.e., gene activation). As used herein, regulatory elements include, but are not limited to, inducible and non-inducible promoters, enhancers, operators, and other elements known to those skilled in the art that drive and regulate expression. Such regulatory elements include, but are not limited to, the cytomegalovirus (hCMV) immediate early gene, regulatable, viral elements (particularly retroviral LTR promoters), the early or late promoters of SV40 or adenovirus, the lac system, the trp system, the TAC system, the TRC system, the major operator and promoter regions of phage lambda, the control regions of fd coat protein, the promoter for 3-phosphoglycerate kinase (PGK), the promoters of acid phosphatase, and the promoters of the yeast α-mating factors.

The present invention also encompasses antibodies and anti-idiotypic antibodies (including Fab fragments), antagonists and agonists of an NHP, as well as compounds or nucleotide constructs that inhibit expression of an NHP sequence (transcription factor inhibitors, antisense and ribozyme molecules, or open reading frame sequence or regulatory sequence replacement constructs), or promote the expression of an NHP (e.g., expression constructs in which NHP coding sequences are operatively associated with expression control elements such as promoters, promoter/enhancers, etc.).

The NHPs or NHP peptides, NHP fusion proteins, NHP nucleotide sequences, antibodies, antagonists and agonists can be useful for the detection of mutant NHPS, or inappropriately expressed NHPs, for the diagnosis of disease. The NHP proteins or peptides, NHP fusion proteins, NHP nucleotide sequences, host cell expression systems, antibodies, antagonists, agonists and genetically engineered cells and animals can be used for screening for drugs (or high throughput screening of combinatorial libraries) effective in the treatment of the symptomatic or phenotypic manifestations of perturbing the normal function of an NHP in the body. The use of engineered host cells and/or animals may offer an advantage in that such systems allow not only for the identification of compounds that bind to an endogenous receptor for an NHP, but can also identify compounds that trigger NHP-mediated activities or pathways.

Finally, the NHP products can be used as therapeutics. For example, soluble derivatives, such as NHP peptides/domains corresponding to an NHP, NHP fusion protein products (especially NHP-Ig fusion proteins, i.e., fusions of an NHP, or a domain of an NHP, to an IgFc), NHP antibodies and anti-idiotypic antibodies (including Fab fragments), antagonists or agonists (including compounds that modulate or act on downstream targets in an NHP-mediated pathway), can be used to directly treat diseases or disorders. For instance, the administration of an effective amount of a soluble NHP, an NHP-IgFc fusion protein, or an anti-idiotypic antibody (or its Fab) that mimics an NHP, could activate or effectively antagonize an endogenous NHP receptor. Nucleotide constructs encoding such NHP products can be used to genetically engineer host cells to express such products in vivo; these genetically engineered cells function as “bioreactors” in the body delivering a continuous supply of an NHP, an NHP peptide, or an NHP fusion protein to the body. Nucleotide constructs encoding functional NHPs, mutant NHPs, as well as antisense and ribozyme molecules, can also be used in “gene therapy” approaches for the modulation of NHP expression. Thus, the invention also encompasses pharmaceutical formulations and methods for treating biological disorders.

Various aspects of the invention are described in greater detail in the subsections below.

### 7.1 NHP Nucleic Acid Sequences

The cDNA sequences and corresponding deduced amino acid sequences of the described NHPs (SEQ ID NOS:1-222) are presented in the Sequence Listing.

A number of polymorphisms were identified during the sequencing of the disclosed nucleic acid sequences, including: an A to G transition at nucleotide (nt) position 1474 of SEQ ID NOS:1 and 3, and nt position 1363 of SEQ ID NOS:5 and 7, which can result in a lysine or glutamate residue at corresponding amino acid (aa) position 492 of SEQ ID NOS:2 and 4, and aa position 455 of SEQ ID NOS:6 and 8; a C to T transition at nt position 1669 of SEQ ID NOS:1 and 3, and nt position 1558 of SEQ ID NOS:5 and 7, which can result in a glutamine residue or stop codon at corresponding aa position 557 of SEQ ID NOS:2 and 4, and aa position 520 of SEQ ID NOS:6 and 8; a T to A transversion at nt position 1673 of SEQ ID NOS:1 and 3, and nt position 1562 of SEQ ID NOS:5 and 7, which can result in a leucine or histidine residue at corresponding aa position 558 of SEQ ID NOS:2 and 4, and aa position 521 of SEQ ID NOS:6 and 8; a T to C transition at nt position 1007 of SEQ ID NOS:23, 27, and 31, and nt position 896 of SEQ ID NOS:25, 29, and 33, which can result in a leucine or serine residue at corresponding aa position 336 of SEQ ID NOS:24, 28, and 32, and aa position 299 of SEQ ID NOS:26, 30, and 34; a G to T transversion at nt position 1014 of SEQ ID NOS:23, 27, and 31, and nt position 903 of SEQ ID NOS:25, 29, and 33, which can result in a glutamate or aspartate residue at corresponding aa position 338 of SEQ ID NOS:24, 28, and 32, and aa position 301 of SEQ ID NOS:26, 30, and 34; a translationally silent T to C transition at nt position 1158 of SEQ ID NO:27; a translationally silent G to A transition at nt position 24 of SEQ ID NO:35 (denoted by an “r” in the Sequence Listing); a G/A polymorphism at nt position 68 of SEQ ID NO:63 and nt position 56 of SEQ ID NO:65 (denoted by an “r” in the Sequence Listing), which can result in an arginine or glutamine residue at corresponding aa position 23 of SEQ ID NO:64 and aa position 19 of SEQ ID NO:66; an A/G polymorphism at nt position 82 of SEQ ID NO:63 and nt position 70 of SEQ ID NO:65 (denoted by an “r” in the Sequence Listing), which can result in an alanine or threonine residue at corresponding aa position 28 of SEQ ID NO:64 and aa position 24 of SEQ ID NO:66; a translationally silent T/C polymorphism at nt position 28 of SEQ ID NO:72 (denoted by an “y” in the Sequence Listing); a T/C polymorphism at nt position 55 of SEQ ID NO:72 (denoted by an “y” in the Sequence Listing), which can result in a tyrosine or histidine residue at corresponding aa 19 of SEQ ID NO:73; a G/A polymorphism at nt position 379 of SEQ ID NO:72 and nt position 199 of SEQ ID NO:74 (denoted by an “r” in the Sequence Listing), which can result in an alanine or threonine residue at corresponding aa position 127 of SEQ ID NO:73 and aa position 67 of SEQ ID NO:75; a translationally silent C/T polymorphism at nt position 951 of SEQ ID NO:121 (denoted by a “y” in the Sequence Listing); a C/T polymorphism at nt position 2,110 of SEQ ID NO:121 (denoted by a “y” in the Sequence Listing), which can result in a proline or serine residue at corresponding aa position 704 of SEQ ID NO:122; a G/A transition at nt position 343 of SEQ ID NO:124 (denoted by an “r” in the Sequence Listing), which can result in a valine or isoleucine residue at corresponding aa position 115 of SEQ ID NO:125; a C/T transition at nt position 868 of SEQ ID NO:124 (denoted by a “y” in the Sequence Listing), which can result in a cysteine or arginine residue at corresponding aa position 290 of SEQ ID NO:125; an AA/GT polymorphism at nt positions 364 and 365 of SEQ ID NOS:127 and 131 (denoted by an “rw” in the Sequence Listing), which can result in an asparagine or valine residue at corresponding aa position 122 of SEQ ID NOS:128 and 132; an A/G polymorphism at nt position 535 of SEQ ID NOS:127 and 131 (denoted by an “r” in the Sequence Listing), which can result in a lysine or glutamate residue at corresponding aa position 179 of SEQ ID NOS:128 and 132; an A/G polymorphism at nt position 58 of SEQ ID NOS:140, 142, 144, 146, 148, 150, 152, 154, 156, and 158 (denoted by an “r” in the Sequence Listing), which can result in a threonine or alanine residue at corresponding aa position 20 of SEQ ID NOS:141, 143, 145, 147, 149, 151, 153, 155, 157, and 159; a CATT/GTCA polymorphism at nt positions 1114-1117 of SEQ ID NO:146 (denoted by an “swyw” in the Sequence Listing), which can result in a valine/threonine or histidine/serine dyad at corresponding aa positions 372 and 373 of SEQ ID NO:147; an A/C polymorphism at nt position 1538 of SEQ ID NOS:144, 146, 148, 150, 152, 154, 156, and 158 (denoted by an “m” in the Sequence Listing), which can result in a proline or glutamine residue at corresponding aa position 513 of SEQ ID NOS:145, 147, 149, 151, 153, 155, 157, and 159; a translationally silent T/C polymorphism at nt position 1542 of SEQ ID NOS:144, 146, 148, 150, 152, 154, 156, and 158 (denoted by an “y” in the Sequence Listing); a T/C polymorphism at nt position 1769 of SEQ ID NOS:144, 146, 148, 150, 152, 154, 156, and 158 (denoted by an “y” in the Sequence Listing), which can result in a proline or leucine residue at corresponding aa position 590 of SEQ ID NOS:145, 147, 149, 151, 153, 155, 157, and 159; a translationally silent C/G polymorphism at nt position 1869 of SEQ ID NOS:144, 146, 148, 150, 152, 154, 156, and 158 (denoted by an “s” in the Sequence Listing); a G/C polymorphism at nt position 1899 of SEQ ID NOS:144, 146, 148, 150, 152, 154, 156, and 158 (denoted by an “s” in the Sequence Listing), which can result in a serine or arginine residue at corresponding aa position 633 of SEQ ID NOS:145, 147, 149, 151, 153, 155, 157, and 159; a translationally silent A/G polymorphism at nt position 1965 of SEQ ID NOS:144, 146, 148, 150, 152, 154, 156, and 158 (denoted by an “r” in the Sequence Listing); a CATT/GTCA polymorphism at nt positions 2259-2262 of SEQ ID NOS:144, 146, 148, 150, 152, 154, 156, and 158 (denoted by an “swyw” in the Sequence Listing), which is translationally silent at corresponding aa position 753, and can result in a serine or isoleucine residue at corresponding aa position 754, of SEQ ID NOS:145, 147, 149, 151, 153, 155, 157, and 159; a G/A polymorphism at nt position 3050 of SEQ ID NOS:144, 146, 148, 150, 152, 154, 156, and 158 (denoted by an “r” in the Sequence Listing), which can result in a serine or asparagine residue at corresponding aa position 1017 of SEQ ID NOS:145, 147, 149, 151, 153, 155, 157, and 159; a G/A polymorphism at nt position 3146 of SEQ ID NOS:144, 146, 148, 150, 152, 154, 156, and 158 (denoted by an “r” in the Sequence Listing), which can result in a glycine or glutamate residue at corresponding aa position 1049 of SEQ ID NOS:145, 147, 149, 151, 153, 155, 157, and 159; an A/T polymorphism at nt position 286 of SEQ ID NO:161 (denoted by a “w” in the Sequence Listing), which can result in a serine or threonine residue at corresponding aa position 96 of SEQ ID NO:162; a translationally silent T/C polymorphism at nt position 3735 of SEQ ID NO:161 (denoted by a “y” in the Sequence Listing); a G/C polymorphism at nt position 491 of SEQ ID NOS:164, 166, and 168, which can result in a glycine or alanine residue at corresponding aa position 164 of SEQ ID NOS:165, 167, and 169; a T/G polymorphism at nt position 2598 of SEQ ID NO:166, which can result in a cysteine or tryptophan residue at corresponding aa position 866 of SEQ ID NO:167; an A/G polymorphism at nt position 838 of SEQ ID NOS:171 and 173, which can result in a threonine or alanine residue at corresponding aa position 280 of SEQ ID NOS:172 and 174; an A/T polymorphism at nt position 1006 of SEQ ID NOS:171 and 173, which can result in a threonine or serine residue at corresponding aa position 336 of SEQ ID NOS:172 and 174; a G/C polymorphism at nt position 1019 of SEQ ID NOS:171 and 173, which can result in a glycine or alanine residue at corresponding aa position 340 of SEQ ID NOS:172 and 174; an A/G polymorphism at nt position 1046 of SEQ ID NOS:171 and 173, which can result in a glutamine or arginine residue at corresponding aa position 349 of SEQ ID NOS:172 and 174; a G/C polymorphism at nt position 149 of SEQ ID NOS:176 and 182, which can result in an arginine or proline residue at corresponding aa position 50 of SEQ ID NOS:177 and 183; a G/C polymorphism at nt position 176 of SEQ ID NOS:176 and 182, which can result in a glycine or alanine residue at corresponding aa position 59 of SEQ ID NOS:177 and 183; a G/C polymorphism at nt position 179 of SEQ ID NOS:176 and 182, which can result in a serine or threonine residue at corresponding aa position 60 of SEQ ID NOS:177 and 183; a G/T polymorphism at nt position 209 of SEQ ID NOS:176 and 182, which can result in an arginine or leucine residue at corresponding aa position 70 of SEQ ID NOS:177 and 183; an A/G polymorphism at nt position 313 of SEQ ID NOS:189, 191, 193, and 195, which can result in a threonine or alanine residue at corresponding aa position 105 of SEQ ID NOS:190, 192, 194 and 196; an A/T polymorphism at nt position 1670 of SEQ ID NOS:189, 191, 193, and 195, which can result in an aspartate or valine residue at corresponding aa position 557 of SEQ ID NOS:190, 192, 194, and 196; an A/G polymorphism at nt position 1864 of SEQ ID NOS:189, 191, 193, and 195, which can result in an aspartate or asparagine residue at corresponding aa position 622 of SEQ ID NOS:190, 192, 194, and 196; a T/C polymorphism at nt position 1915 of SEQ ID NOS:189, 191, 193, and 195, which can result in a phenylalanine or leucine residue at corresponding aa position 639 of SEQ ID NOS:190, 192, 194, and 196; a G/A polymorphism at nt position 445 of SEQ ID NOS:198 and 200, which can result in a valine or isoleucine residue at corresponding aa position 149 of SEQ ID NOS:199 and 201; a C/T polymorphism at nt position 457 of SEQ ID NOS:198 and 200, which can result in an arginine or tryptophan residue at corresponding aa position 153 of SEQ ID NOS:199 and 201; an A/G polymorphism at nt position 4079 of SEQ ID NO:202, nt position 4454 of SEQ ID NO:204, and nt position 4502 of SEQ ID NO:206, which can result in either a lysine or arginine residue at corresponding aa position 1360 of SEQ ID NO:203, aa position 1485 of SEQ ID NO:205, and aa position 1501 of SEQ ID NO:207; a C/G polymorphism at nt position 2361 of SEQ ID NO:212, which can result in an aspartate or glutamate residue at corresponding aa position 787 of SEQ ID NO:213; a C/A polymorphism at nt position 2467 of SEQ ID NO:212, which can result in a leucine or isoleucine residue at corresponding aa position 823 of SEQ ID NO:213; a translationally silent C/A polymorphism at nt position 2613 of SEQ ID NO:212; a translationally silent C/T polymorphism at nt position 3141 of SEQ ID NO:212; a G/T polymorphism at nt position 3225 of SEQ ID NO:212, which can result in a glutamine or histidine residue at corresponding aa position 1075 of SEQ ID NO:213; a C/T polymorphism at nt position 3226 of SEQ ID NO:212, which can result in an arginine or tryptophan residue at corresponding aa position 1076 of SEQ ID NO:213; and an A/G polymorphism at nt position 4226 of SEQ ID NO:212, which can result in an aspartate or glycine residue at corresponding aa position 1409 of SEQ ID NO:213.

The described NHPs are apparently encoded on: human chromosome 2, see GenBank Accession No. AC092569 (SEQ ID NOS:171-175); human chromosome 5, see GenBank Accession No. AC008528 SEQ ID NOS:176-188); human chromosome 5, see GenBank Accession No. AC008676 (SEQ ID NOS:189-197); human chromosome 1, see, e.g., GenBank Accession No. AL365208 (SEQ ID NOS:198-201); human chromosome 2, see GenBank Accession No. AC011231 (SEQ ID NOS:202-208); human chromosome 2 (SEQ ID NOS:202-208); human chromosome 1, see GenBank Accession No. AL356356 (SEQ ID NOS:209-211); human chromosome 9, see GenBank Accession No. AL158150 (SEQ ID NOS:212 and 213); human chromosome 2, see, for example, GenBank Accession No. AC012307 (SEQ ID NOS:214-216); and human chromosome 7 and/or 16, see GenBank Accession Nos. AC025284 and AC026498 (SEQ ID NOS:217-222).

An additional application of the described novel human polynucleotide sequences is their use in the molecular mutagenesis/evolution of proteins that are at least partially encoded by the described novel sequences using, for example, polynucleotide shuffling or related methodologies (see, e.g., U.S. Pat. Nos. 5,830,721 and 5,837,458).

NHP gene products can also be expressed in transgenic animals. Animals of any non-human species, including, but not limited to, worms, mice, rats, rabbits, guinea pigs, pigs, micro-pigs, birds, goats, and non-human primates, e.g., baboons, monkeys, and chimpanzees, may be used to generate NHP transgenic animals.

Any technique known in the art may be used to introduce an NHP transgene into animals to produce the founder lines of transgenic animals. Such techniques include, but are not limited to: pronuclear microinjection (U.S. Pat. No. 4,873,191); retrovirus-mediated gene transfer into germ lines (Van der Putten et al., Proc. Natl. Acad. Sci. USA 82:6148-6152, 1985); gene targeting in embryonic stem cells (Thompson et al., Cell 56:313-321, 1989); electroporation of embryos (Lo, Mol. Cell. Biol. 3:1803-1814, 1983); sperm-mediated gene transfer (Lavitrano et al., Cell 57:717-723, 1989); and positive-negative selection, as described in U.S. Pat. No. 5,464,764. For a review of such techniques, see Gordon, Intl. Rev. Cytol. 115:171-229, 1989.

The present invention provides for non-human transgenic animals that carry an NHP transgene in all their cells, as well as animals that carry an NHP transgene in some, but not all their cells, i.e., mosaic animals or somatic cell transgenic animals. Animals of any species, including, but not limited to, mice, rats, rabbits, guinea pigs, pigs, micro-pigs, goats, and non-human primates, e.g., baboons, monkeys, and chimpanzees, can be used to generate transgenic animals carrying NHP polynucleotides. NHP transgenes may be integrated as a single transgene or in concatamers, e.g., head-to-head tandems or head-to-tail tandems. The transgene may also be selectively introduced into and activated in a particular cell type by following, for example, the teaching of Lasko et al., Proc. Natl. Acad. Sci. USA 89:6232-6236, 1992. The regulatory sequences required for such a cell-type specific activation will depend upon the particular cell type of interest, and will be apparent to those of skill in the art.

When it is desired that an NHP transgene be integrated into the chromosomal site of the endogenous NHP gene, gene targeting is preferred. Briefly, when such a technique is to be utilized, vectors containing some nucleotide sequences homologous to the endogenous NHP gene are designed for the purpose of integrating, via homologous recombination with chromosomal sequences, into and disrupting the function of the nucleotide sequence of the endogenous NHP gene (i.e., “knockout” animals).

The transgene can also be selectively introduced into a particular cell-type, thus inactivating the endogenous NHP gene in only that cell-type, by following, for example, the teaching of Gu et al., Science 265:103-106, 1994. The regulatory sequences required for such a cell-type specific inactivation will depend upon the particular cell-type of interest, and will be apparent to those of skill in the art.

Once transgenic animals have been generated, the expression of the recombinant NHP gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques that include, but are not limited to, Northern blot analysis of tissue samples obtained from the animal, in situ hybridization analysis, and RT-PCR. Samples of NHP gene-expressing tissue may also be evaluated immunocytochemically using antibodies specific for the NHP transgene product.

The present invention also provides for “knock-in” animals. Knock-in animals are those in which a polynucleotide sequence (i.e., a gene or a cDNA) that the animal does not naturally have in its genome is inserted in such a way that it is expressed. Examples include, but are not limited to, a human gene or cDNA used to replace its murine ortholog in the mouse, a murine cDNA used to replace the murine gene in the mouse, and a human gene or cDNA or murine cDNA that is tagged with a reporter construct used to replace the murine ortholog or gene in the mouse. Such replacements can occur at the locus of the murine ortholog or gene, or at another specific site. Such knock-in animals are useful for the in vivo study, testing and validation of, intra alia, human drug targets, as well as for compounds that are directed at the same, and therapeutic proteins.

### 7.2 NHP Amino Acid Sequences

NHPs, NHP polypeptides, NHP peptide fragments, mutated, truncated, or deleted forms of NHPs, and/or NHP fusion proteins can be prepared for a variety of uses. These uses include, but are not limited to, the generation of antibodies, as therapeutics (for treating abnormal levels of white blood cells, cardiovascular disease, stenosis (or preventing restenosis), inflammatory or proliferative disorders, infectious disease, cancer, etc.), as reagents in diagnostic assays, for the identification of other cellular gene products related to the NHPs, as reagents in assays for screening for compounds that can be used as pharmaceutical reagents useful in the therapeutic treatment of mental, biological, or medical disorders and disease. Given the similarity information and expression data, the described NHPs can be targeted (by drugs, oligos, antibodies, etc.) in order to treat disease, or to augment the efficacy of therapeutic agents.

The Sequence Listing discloses the amino acid sequences encoded by the described NHP polynucleotides. The NHPs display initiator methionines in DNA sequence contexts consistent with a translation initiation site. Some of the NHPs display signal sequences, which can indicate that such NHPs may be secreted or membrane associated, while other NHPs do not display a consensus signal sequence, which can indicate that such NHP ORFs can be exemplary of the mature or processed forms of the NHPs as typically found in the body. The sequence data presented herein indicate that alternatively spliced forms of the NHPs exist (which may or may not be tissue specific).

The NHP amino acid sequences of the invention include the amino acid sequences presented in the Sequence Listing, as well as analogues and derivatives thereof. Further, corresponding NHP homologues from other species are encompassed by the invention. In fact, any NHP encoded by the NHP nucleotide sequences described herein are within the scope of the invention, as are any novel polynucleotide sequences encoding all or any novel portion of an amino acid sequence presented in the Sequence Listing. The degenerate nature of the genetic code is well-known, and, accordingly, each amino acid presented in the Sequence Listing is generically representative of the well-known nucleic acid “triplet” codon, or in many cases codons, that can encode the amino acid. As such, as contemplated herein, the amino acid sequences presented in the Sequence Listing, when taken together with the genetic code (see, for example, “Molecular Cell Biology”, Table 4-1 at page 109 (Darnell et al., eds., Scientific American Books, New York, N.Y., 1986)), are generically representative of all the various permutations and combinations of nucleic acid sequences that can encode such amino acid sequences.

The invention also encompasses proteins that are functionally equivalent to the NHPs encoded by the presently described nucleotide sequences as judged by any of a number of criteria, including, but not limited to, the ability to bind and cleave a substrate of an NHP, or the ability to effect an identical or complementary downstream pathway, or a change in cellular metabolism (e.g., proteolytic activity, ion flux, tyrosine phosphorylation, etc.). Such functionally equivalent NHP proteins include, but are not limited to, additions or substitutions of amino acid residues within the amino acid sequence encoded by the NHP nucleotide sequences described herein, but that result in a silent change, thus producing a functionally equivalent expression product. Amino acid substitutions can be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

A variety of host-expression vector systems can be used to express the NHP nucleotide sequences of the invention. Where, as in the present instance, the NHP peptides or polypeptides are thought to be soluble or secreted molecules, the peptides or polypeptides can be recovered from the culture media. Such expression systems also encompass engineered host cells that express an NHP, or a functional equivalent, in situ. Purification or enrichment of an NHP from such expression systems can be accomplished using appropriate detergents and lipid micelles and methods well-known to those skilled in the art. However, such engineered host cells themselves may be used in situations where it is important not only to retain the structural and functional characteristics of an NHP, but to assess biological activity, e.g., in certain drug screening assays.

The expression systems that may be used for purposes of the invention include, but are not limited to, microorganisms such as bacteria (e.g., E. coli, B. subtilis) transformed with recombinant bacteriophage DNA, plasmid DNA or cosmid DNA expression vectors containing NHP nucleotide sequences; yeast (e.g., Saccharomyces, Pichia) transformed with recombinant yeast expression vectors containing NHP nucleotide sequences; insect cell systems infected with recombinant virus expression vectors (e.g., baculovirus) containing NHP nucleotide sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing NHP nucleotide sequences; or mammalian cell systems (e.g., COS, CHO, BHK, 293, 3T3) harboring recombinant expression constructs containing NHP nucleotide sequences and promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the NHP product being expressed. For example, when a large quantity of such a protein is to be produced for the generation of pharmaceutical compositions of or containing an NHP, or for raising antibodies to an NHP, vectors that direct the expression of high levels of fusion protein products that are readily purified may be desirable. Such vectors include, but are not limited to, the E. coli expression vector pUR278 (Ruther and Muller-Hill, EMBO J. 2:1791-1794, 1983), in which an NHP coding sequence may be ligated individually into the vector in-frame with the lacZ coding region so that a fusion protein is produced, pIN vectors (Inouye and Inouye, Nucl. Acids Res. 13:3101-3109, 1985; Van Heeke and Schuster, J. Biol. Chem. 264:5503-5509, 1989), and the like. pGEX vectors (Pharmacia or American Type Culture Collection) can also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target expression product can be released from the GST moiety.

In an exemplary insect system, Autographa californica nuclear polyhedrosis virus (AcNPV) is used as a vector to express foreign polynucleotide sequences. The virus grows in Spodoptera frugiperda cells. An NHP coding sequence can be cloned individually into a non-essential region (for example the polyhedrin gene) of the virus and placed under control of an AcNPV promoter (for example the polyhedrin promoter). Successful insertion of an NHP coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted sequence is expressed (see, e.g., Smith et al., J. Virol. 46:584-593, 1983, and U.S. Pat. No. 4,215,051).

In mammalian host cells, a number of viral-based expression systems may be utilized. In cases where an adenovirus is used as an expression vector, the NHP nucleotide sequence of interest may be ligated to an adenovirus transcription/translation control complex, e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing an NHP product in infected hosts (see, e.g., Logan and Shenk, Proc. Natl. Acad. Sci. USA 81:3655-3659, 1984). Specific initiation signals may also be required for efficient translation of inserted NHP nucleotide sequences. These signals include the ATG initiation codon and adjacent sequences. In cases where an entire NHP gene or cDNA, including its own initiation codon and adjacent sequences, is inserted into the appropriate expression vector, no additional translational control signals may be needed. However, in cases where only a portion of an NHP coding sequence is inserted, exogenous translational control signals, including, perhaps, the ATG initiation codon, may be provided. Furthermore, the initiation codon should be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bitter et al., Methods in Enzymol. 153:516-544, 1987).

In addition, a host cell strain may be chosen that modulates the expression of the inserted sequences, or modifies and processes the expression product in the specific fashion desired. Such modifications (e.g., glycosylation) and processing (e.g., cleavage) of protein products may be important for the function of the protein. Different host cells have characteristic and specific mechanisms for the post-translational processing and modification of proteins and expression products. Appropriate cell lines or host systems can be chosen to ensure the desired modification and processing of the foreign protein expressed. To this end, eukaryotic host cells that possess the cellular machinery for the desired processing of the primary transcript, glycosylation, and phosphorylation of the expression product may be used. Such mammalian host cells include, but are not limited to, CHO, VERO, BHK, HeLa, COS, MDCK, 293, 3T3, WI38, and in particular, human cell lines.

For long-term, high-yield production of recombinant proteins, stable expression is preferred. For example, cell lines that stably express the NHP sequences described herein can be engineered. Rather than using expression vectors that contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then switched to a selective media. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines that express an NHP product. Such engineered cell lines may be particularly useful in screening and evaluation of compounds that affect the endogenous activity of an NHP product.

A number of selection systems may be used, including, but not limited to, the herpes simplex virus thymidine kinase (Wigler et al., Cell 11:223-232, 1977), hypoxanthine-guanine phosphoribosyltransferase (Szybalska and Szybalski, Proc. Natl. Acad. Sci. USA 48:2026-2034, 1962), and adenine phosphoribosyltransferase (Lowy et al., Cell 22:817-823, 1980) genes, which can be employed in tk⁻, hgprt⁻ or aprt⁻ cells, respectively. Also, antimetabolite resistance can be used as the basis of selection for the following genes: dihydrofolate reductase (dhfr), which confers resistance to methotrexate (Wigler et al., Proc. Natl. Acad. Sci. USA 77:3567-3570, 1980, and O'Hare et al., Proc. Natl. Acad. Sci. USA 78:1527-1531, 1981); guanine phosphoribosyl transferase (gpt), which confers resistance to mycophenolic acid (Mulligan and Berg, Proc. Natl. Acad. Sci. USA 78:2072-2076, 1981); neomycin phosphotransferase (neo), which confers resistance to G-418 (Colbere-Garapin et al., J. Mol. Biol. 150:1-14, 1981); and hygromycin B phosphotransferase (hpt), which confers resistance to hygromycin (Santerre et al., Gene 30:147-156, 1984).

Alternatively, any fusion protein can be readily purified by utilizing an antibody specific for the fusion protein being expressed. Another exemplary system allows for the ready purification of non-denatured fusion proteins expressed in human cell lines (Janknecht et al., Proc. Natl. Acad. Sci. USA 88:8972-8976, 1991). In this system, the sequence of interest is subcloned into a vaccinia recombination plasmid such that the sequence's open reading frame is translationally fused to an amino-terminal tag consisting of six histidine residues. Extracts from cells infected with recombinant vaccinia virus are loaded onto Ni²⁺.nitriloacetic acid-agarose columns, and histidine-tagged proteins are selectively eluted with imidazole-containing buffers.

Also encompassed by the present invention are fusion proteins that direct an NHP to a target organ and/or facilitate transport across the membrane into the cytosol. Conjugation of NHPs to antibody molecules or their Fab fragments could be used to target cells bearing a particular epitope. Attaching an appropriate signal sequence to an NHP would also transport an NHP to a desired location within the cell. Alternatively targeting of an NHP or its nucleic acid sequence might be achieved using liposome or lipid complex based delivery systems. Such technologies are described in “Liposomes: A Practical Approach” (New, R. R. C., ed., IRL Press, New York, N.Y., 1990), and in U.S. Pat. Nos. 4,594,595, 5,459,127, 5,948,767 and 6,110,490. Additionally embodied are novel protein constructs engineered in such a way that they facilitate transport of NHPs to a target site or desired organ, where they cross the cell membrane and/or the nucleus where the NHPs can exert their functional activity. This goal may be achieved by coupling of an NHP to a cytokine or other ligand that provides targeting specificity, and/or to a protein transducing domain (see generally U.S. Provisional Patent Application Ser. Nos. 60/111,701 and 60/056,713, for examples of such transducing sequences), to facilitate passage across cellular membranes, and can optionally be engineered to include nuclear localization signals.

Additionally contemplated are oligopeptides that are modeled on an amino acid sequence first described in the Sequence Listing. Such NHP oligopeptides are generally between about 10 to about 100 amino acids long, or between about 16 to about 80 amino acids long, or between about 20 to about 35 amino acids long, or any variation or combination of sizes represented therein that incorporate a contiguous region of sequence first disclosed in the Sequence Listing. Such NHP oligopeptides can be of any length disclosed within the above ranges and can initiate at any amino acid position represented in the Sequence Listing.

The invention also contemplates “substantially isolated” or “substantially pure” proteins or polypeptides. By a “substantially isolated” or “substantially pure” protein or polypeptide is meant a protein or polypeptide that has been separated from at least some of those components that naturally accompany it. Typically, the protein or polypeptide is substantially isolated or pure when it is at least 60%, by weight, free from the proteins and other naturally-occurring organic molecules with which it is naturally associated in vivo. Preferably, the purity of the preparation is at least 75%, more preferably at least 90%, and most preferably at least 99%, by weight. A substantially isolated or pure protein or polypeptide may be obtained, for example, by extraction from a natural source, by expression of a recombinant nucleic acid encoding the protein or polypeptide, or by chemically synthesizing the protein or polypeptide.

Purity can be measured by any appropriate method, e.g., column chromatography such as immunoaffinity chromatography using an antibody specific for the protein or polypeptide, polyacrylamide gel electrophoresis, or HPLC analysis. A protein or polypeptide is substantially free of naturally associated components when it is separated from at least some of those contaminants that accompany it in its natural state. Thus, a polypeptide that is chemically synthesized or produced in a cellular system different from the cell from which it naturally originates will be, by definition, substantially free from its naturally associated components. Accordingly, substantially isolated or pure proteins or polypeptides include eukaryotic proteins synthesized in E. coli, other prokaryotes, or any other organism in which they do not naturally occur.

### 7.3 Antibodies to NHP Products

Antibodies that specifically recognize one or more epitopes of an NHP, epitopes of conserved variants of an NHP, or peptide fragments of an NHP, are also encompassed by the invention. Such antibodies include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

The antibodies of the invention may be used, for example, in the detection of an NHP in a biological sample and may, therefore, be utilized as part of a diagnostic or prognostic technique whereby patients may be tested for abnormal amounts of an NHP. Such antibodies may also be utilized in conjunction with, for example, compound screening schemes for the evaluation of the effect of test compounds on expression and/or activity of an NHP expression product. Additionally, such antibodies can be used in conjunction with gene therapy to, for example, evaluate normal and/or engineered NHP-expressing cells prior to their introduction into a patient. Such antibodies may additionally be used in methods for the inhibition of abnormal NHP activity. Thus, such antibodies may be utilized as a part of treatment methods.

For the production of antibodies, various host animals may be immunized by injection with an NHP, an NHP peptide (e.g., one corresponding to a functional domain of an NHP), a truncated NHP polypeptide (an NHP in which one or more domains have been deleted), functional equivalents of an NHP or mutated variants of an NHP. Such host animals may include, but are not limited to, pigs, rabbits, mice, goats, and rats, to name but a few. Various adjuvants may be used to increase the immunological response, depending on the host species, including, but not limited to, Freund's adjuvant (complete and incomplete), mineral salts such as aluminum hydroxide or aluminum phosphate, chitosan, surface active substances such as lysolecithin, pluronic polyols, polyanions, peptides, oil emulsions, and potentially useful human adjuvants such as BCG (bacille Calmette-Guerin) and Corynebacterium parvum. Alternatively, the immune response could be enhanced by combination and/or coupling with molecules such as keyhole limpet hemocyanin, tetanus toxoid, diphtheria toxoid, ovalbumin, cholera toxin, or fragments thereof. Polyclonal antibodies are heterogeneous populations of antibody molecules derived from the sera of the immunized animals.

Monoclonal antibodies, which are homogeneous populations of antibodies to a particular antigen, can be obtained by any technique that provides for the production of antibody molecules by continuous cell lines in culture. These include, but are not limited to, the hybridoma technique (Kohler and Milstein, Nature 256:495-497, 1975, and U.S. Pat. No. 4,376,110), the human B-cell hybridoma technique (Kosbor et al., Immunology Today 4:72, 1983, and Cole et al., Proc. Natl. Acad. Sci. USA 80:2026-2030, 1983), and the EBV-hybridoma technique (Cole et al., in “Monoclonal Antibodies and Cancer Therapy”, Vol. 27, UCLA Symposia on Molecular and Cellular Biology, New Series, pp. 77-96 (Reisfeld and Sell, eds., Alan R. Liss, Inc. New York, N.Y., 1985)). Such antibodies may be of any immunoglobulin class, including IgG, IgM, IgE, IgA, and IgD, and any subclass thereof. The hybridomas producing the mAbs of this invention may be cultivated in vitro or in vivo. Production of high titers of mabs in vivo makes this the presently preferred method of production.

In addition, techniques developed for the production of “chimeric antibodies” (Morrison et al., Proc. Natl. Acad. Sci. USA 81:6851-6855, 1984., Neuberger et al., Nature 312:604-608, 1984, and Takeda et al., Nature 314:452-454, 1985) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. Such technologies are described in U.S. Pat. Nos. 6,114,598, 6,075,181 and 5,877,397. Also encompassed by the present invention is the use of fully humanized monoclonal antibodies, as described in U.S. Pat. No. 6,150,584.

Alternatively, techniques described for the production of single chain antibodies (U.S. Pat. No. 4,946,778, Bird, Science 242:423-426, 1988, Huston et al., Proc. Natl. Acad. Sci. USA 85:5879-5883, 1988, and Ward et al., Nature 341:544-546, 1989) can be adapted to produce single chain antibodies against NHP expression products. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include, but are not limited to: F(ab′)₂ fragments, which can be produced by pepsin digestion of an antibody molecule; and Fab fragments, which can be generated by reducing the disulfide bridges of F(ab′)₂ fragments. Alternatively, Fab expression libraries may be constructed (Huse et al., Science 246:1275-1281, 1989) to allow rapid and easy identification of monoclonal Fab fragments with the desired specificity.

Antibodies to an NHP can, in turn, be utilized to generate anti-idiotype antibodies that “mimic” a given NHP, using techniques well-known to those skilled in the art (see, e.g., Greenspan and Bona, FASEB J. 7:437-444, 1993, and Nissinoff, J. Immunol. 147:2429-2438, 1991). For example, antibodies that bind to an NHP domain and competitively inhibit the binding of an NHP to its cognate receptor can be used to generate anti-idiotypes that “mimic” the NHP and, therefore, bind and activate or neutralize a receptor. Such anti-idiotypic antibodies, or Fab fragments of such anti-idiotypes, can be used in therapeutic regimens involving an NHP signaling pathway.

Additionally given the high degree of relatedness of mammalian NHPs, NHP knock-out mice (having never seen an NHP, and thus never been tolerized to an NHP) have an unique utility, as they can be advantageously applied to the generation of antibodies against the disclosed mammalian NHPs (i.e., the NHPs will be immunogenic in NHP knock-out animals).

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein, will become apparent to those skilled in the art from the foregoing description. Such modifications are intended to fall within the scope of the appended claims. All cited publications, patents, and patent applications are herein incorporated by reference in their entirety.

1. An isolated nucleic acid molecule that encodes the amino acid sequence of SEQ ID NO:2, 4, 6, 8, 11, 13, 15, 17, 19, 21, 24, 26, 28, 30, 32, 34, 36, 39, 41, 43, 45, 47, 49, 51, 53, 55, 57, 59, 61, 64, 66, 68, 71, 73, 75, 78, 80, 82, 84, 86, 88, 90, 92, 94, 96, 98, 100, 102, 105, 107, 109, 111, 113, 115, 117, 119, 122, 125, 128, 130, 132, 134, 136, 139, 141, 143, 145, 147, 149, 151, 153, 155, 157, 159, 162, 165, 167, 169, 172, 174, 177, 179, 181, 183, 185, 187, 190, 192, 194, 196, 199, 201, 203, 205, 207, 210, 213, 215, 218, or 221.

2. The isolated nucleic acid molecule of claim 1, wherein said nucleic acid molecule comprises the nucleotide sequence of SEQ ID NO: 1, 3, 5, 7, 10, 12, 14, 16, 18, 20, 23, 25, 27, 29, 31, 33, 35, 38, 40, 42, 44, 46, 48, 50, 52, 54, 56, 58, 60, 63, 65, 67, 70, 72,74, 77, 79, 81, 83, 85, 87, 89, 91, 93, 95, 97, 99, 101, 104, 106, 108, 110, 112, 114, 116, 118, 121, 124, 127, 129, 131, 133, 135, 138, 140, 142, 144, 146, 148, 150, 152, 154, 156, 158, 161, 164, 166, 168, 171, 173, 176, 178, 180, 182, 184, 186, 189, 191, 193, 195, 198, 200, 202, 204, 206, 209, 212, 214, 217, or 220.

3. An expression vector comprising the isolated nucleic acid molecule of claim 1.

4. (canceled)

5. An isolated polypeptide comprising the amino acid sequence of SEQ ID NO:2, 4, 6, 8, 11, 13, 15, 17, 19, 21, 24, 26, 28, 30, 32, 34, 36, 39, 41, 43, 45, 47, 49, 51, 53, 55, 57, 59, 61, 64, 66, 68, 71, 73, 75, 78, 80, 82, 84, 86, 88, 90, 92, 94, 96, 98, 100, 102, 105, 107, 109, 111, 113, 115, 117, 119, 122, 125, 128, 130, 132, 134, 136, 139, 141, 143, 145, 147, 149, 151, 153, 155, 157, 159, 162, 165, 167, 169, 172, 174, 177, 179, 181, 183, 185, 187, 190, 192, 194, 196, 199, 201, 203, 205, 207, 210, 213, 215, 218, or 221.

6. An antibody that selectively binds a polypeptide drawn from the group consisting of: SEQ ID NO:2, 4, 6, 8, 11, 13, 15, 17, 19, 21, 24, 26, 28, 30, 32, 34, 36, 39, 41, 43, 45, 47, 49, 51, 53, 55, 57, 59, 61, 64, 66, 68, 71, 73, 75, 78, 80, 82, 84, 86, 88, 90, 92, 94, 96, 98, 100, 102, 105, 107, 109, 111, 113, 115, 117, 119, 122, 125, 128, 130, 132, 134, 136, 139, 141, 143, 145, 147, 149, 151, 153, 155, 157, 159, 162, 165, 167, 169, 172, 174, 177, 179, 181, 183, 185, 187, 190, 192, 194, 196, 199, 201, 203, 205, 207, 210, 213, 215, 218, and 221.

7. An oligonucleotide that inhibits the expression of a nucleic acid molecule that encodes an amino acid sequence drawn from the group consisting of SEQ ID NO:2, 4, 6, 8, 11, 13, 15, 17, 19, 21, 24, 26, 28, 30, 32, 34, 36, 39, 41, 43, 45, 47, 49, 51, 53, 55, 57, 59, 61, 64, 66, 68, 71, 73, 75, 78, 80, 82, 84, 86, 88, 90, 92, 94, 96, 98, 100, 102, 105, 107, 109, 111, 113, 115, 117, 119, 122, 125, 128, 130, 132, 134, 136, 139, 141, 143, 145, 147, 149, 151, 153, 155, 157, 159, 162, 165, 167, 169, 172, 174, 177, 179, 181, 183, 185, 187, 190, 192, 194, 196, 199, 201, 203, 205, 207, 210, 213, 215, 218, and 221.
